(19) United States
(12) Patent Application Publication
(10) Pub. No.: US 2008/0274184 A1
(43) Pub. Date: Nov. 6, 2008

(54) Ecm-Based Graft Material

(76) Inventor: James B. Hunt, Bloomington, IN (US)

Correspondence Address: BRINKS HOFER GILSON & LIONE/CHICAGO/COOK, PO BOX 10395, CHICAGO, IL, 60610, US

(21) Appl. No.: 11/547,348

(22) PCT Filed: Mar. 31, 2005
(86) PCT No.: PCT/US05/10905

§ 371 Date: Sep. 29, 2006

Related U.S. Application Data
(60) Provisional application No. 60/558,175, filed on Mar. 31, 2004.

(51) Int. Cl. A61K 9/00; A61K 31/43; A61K 31/545; A61K 31/42; A61K 31/7042; A61K 31/4164; A61K 31/164; A61K 31/65; A61K 31/496; A61K 31/7048; A61K 31/18; A61K 31/505; A61K 31/7076; A61P 17/02

(52) U.S. Cl. 424/484; 514/192; 514/200; 514/380; 514/31; 514/396; 514/628; 514/152; 514/254.11; 514/29; 514/601; 514/274; 514/45

(57) ABSTRACT

This invention is directed to graft materials comprising an extracellular matrix (ECM) and therapeutic agents. This invention is also directed to methods of using the graft materials for healing of damaged or diseased tissues on a patient's body.

### RELATED APPLICATIONS

The present patent document claims the benefit of the filing date under 35 U.S.C. §119(e) of Provisional U.S. Patent Application Ser. No. 60/558,175, filed Mar. 31, 2004, which is hereby incorporated by reference.

### BACKGROUND

1. Technical Field

This invention is directed to graft materials comprising an extracellular matrix (ECM) and therapeutic agents. This invention is also directed to methods of using the graft materials for healing of damaged or diseased tissues on a patient's body.

2. Background Information

Inadequate methods and compositions to effectively heal chronic or temporary wounds is a significant health care problem. Impaired wound healing increases the chances of mortality and morbidity. A skin wound is defined as a breach in the continuity of any body tissue caused by a minimal direct injury to the skin. A quick closure of the wounded skin will promote a beneficial response.

Among the most common injuries to skin are burns. Burns cause destruction of the epidermis and deeper cutaneous and subcutaneous tissues. Most of that tissue can be regenerated by the normal healing response, if the area burned is not extensive or contaminated. Burns cause more than 2 million injuries annually in the U.S.A., and more than 10,000 deaths each year result from serious burn injuries.

Severe, life threatening wounds on body extremities are also common in patients with diabetes. Chronic diabetic foot ulcers often lead to amputations. An effective treatment of such wounds is desired.

It is known that effective repair and regeneration of injured tissues and organs depends on early establishment of the blood flow needed for cellular infiltration and metabolic support. Biomaterials designed to replace damaged or diseased tissues must act as supports (i.e., scaffolds) into which cells can migrate and establish this needed supply (Han Z C and Liu Y, Int. J. Hematol. 70:68 (1999)).

Previously, one approach was to treat damaged or diseased tissues with synthetically derived biocompatible polymer scaffolds to serve as backbones for tissue and repair and regeneration. These synthetic polymer scaffolds are strong and can be fabricated to degrade following deposition at predetermined rates (or not at all). Also, these synthetic scaffolds can be designed to mimic the material properties of the native tissue they are to replace. However, several clinical complications are often encountered when using synthetic scaffolds.

Because of these complications, another approach was to repair and regenerate tissue utilizing intact extracellular matrix (ECM) obtained from animal tissues as the growth support for host cells.

Badylak et al. (Badylak et al., The Heart Surg. Forum #2002-72222 6(2) (2003)) studied the use of porcine ECM scaffolds in connection with repair of the myocardial tissue. Badylak et al. found that both urinary bladder submucosa (UBM) and small intestine submucosa-ECM (SIS-ECM) scaffolds were totally resorbed following surgical implantation and were replaced by a mixture of connective tissue, including cardiac muscle, fibrous connective tissue, adipose connective tissue, and cartilaginous connective tissue.

Bilbo (WO 02/22184) taught tissue engineered multi-layered prostheses made from processed tissue matrices derived from native tissues, intestinal collagen (ICL), that are biocompatible with the patient or host in which they are implanted.

Compositions comprising the tunica submucosa of the intestine of warm-blooded vertebrates can be used as tissue graft materials. Such tissue graft compositions are characterized by excellent mechanical properties, including a high burst pressure, and an effective porosity index which allows such compositions to be used beneficially for vascular graft and connective tissue graft constructs. When used in such applications the graft constructs appear not only to serve as a matrix for the regrowth of the tissues replaced by the graft constructs, but also promote or induce such regrowth of endogenous tissue. Common events to this remodeling process include: widespread and very rapid neovascularization, proliferation of granulation mesenchymal cells, biodegradation/resorption of implanted intestinal submucosal tissue material, and absence of immune rejection.

We here propose novel graft materials and methods for using these graft materials for healing of injured or diseased tissues on a patient's body.

### SUMMARY

In one embodiment, the present invention encompasses a graft material comprising an extracellular matrix (ECM) and at least one therapeutic agent. The ECM of the graft material is preferably an extracellular collagenous matrix. The therapeutic agents present in the graft material may be growth factors, antibiotics, anti-fungal agents, analgesics, antivirals, steroidal anti-inflammatories, non-steroidal anti-inflammatories, anti-neoplastics, anti-spasmodics, modulators of cell-extracellular matrix interactions, enzymes and enzyme inhibitors, anticoagulants and/or anti-thrombotic agents, DNA, RNA, inhibitors of DNA, RNA or protein synthesis, polypeptides, compounds modulating cell migration, compounds modulating proliferation and/or growth, and vasodilating agents.

In another embodiment, the present invention is a method for promoting healing of tissues. The method comprises a step of contacting a tissue in need of healing with a graft material. The graft material includes an ECM and at least one therapeutic agent. The ECM of the graft material is preferably an extracellular collagenous matrix. The therapeutic agents present in the graft material may be growth factors, antibiotics, anti-fungal agents, analgesics, antivirals, steroidal anti-inflammatories, non-steroidal anti-inflammatories, anti-neoplastics, anti-spasmodics, modulators of cell-extracellular matrix interactions, enzymes and enzyme inhibitors, anticoagulants and/or anti-thrombotic agents, DNA, RNA, inhibitors of DNA, RNA or protein synthesis, polypeptides, compounds modulating cell migration, compounds modulating proliferation and/or growth, and vasodilating agents.

### BRIEF DESCRIPTION OF THE DRAWINGS

FIG. 1 is a schematic illustration of the graft material of this invention, wherein the therapeutic agents are added to the ECM after preparation of the ECM.

FIG. 2 is a schematic illustration of the graft material, wherein the therapeutic agents are incorporated into the ECM of the graft material

### DETAILED DESCRIPTION OF THE PRESENTLY PREFERRED EMBODIMENTS

It is to be understood that this invention is not limited to the particular methodology, protocols, cell lines, animal species or genera, constructs, or reagents described and as such may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a,” “an,” and “the” include plural reference unless the context clearly dictates otherwise. Thus, for example, reference to “a cell” is a reference to one or more cells and includes equivalents thereof known to those skilled in the art, and so forth.

Unless defined otherwise, all technical and scientific terms used herein have the same meaning as commonly understood to one of ordinary skill in the art to which this invention belongs. Although any methods, devices, and materials similar or equivalent to those described herein may be used in the practice or testing of the invention, the preferred methods, devices and materials are now described.

The present invention describes graft materials and methods of using of the graft materials for healing of damaged or diseased tissues on a patient body, while delivering therapeutic agents to the patient.

In one embodiment, this present invention contemplates a graft material that includes extracellular matrix (ECM) and at least one therapeutic agent.

In another embodiment, the present invention contemplates a method for promoting healing of tissues. The method comprises contacting a tissue in need of thereof with a graft material. The graft material includes ECM and at least one therapeutic agent.

One advantage of using the graft material of this invention, for example is that it may reduce the necessity for repeated debridement of a part of a patient's body in need of treatment with the graft material.

### DEFINITIONS OF TERMS

“Graft” is a portion of a tissue or organ transplanted from a donor to a recipient to repair a part of a body; in some cases the patient can be both donor and recipient. For example a graft may replace tissue that has been destroyed or create new tissue where none exists.

The term “biocompatible” refers to something, such as certain types of extracellular matrix material, that can be substantially non-toxic in the in vivo environment of its intended use, and is not substantially rejected by the patient's physiological system (i.e., is non-antigenic). This can be gauged by the ability of a material to pass the biocompatibility tests set forth in International Standards Organization (ISO) Standard No. 10993 and/or the U.S. Pharmacopeia (USP) 23 and/or the U.S. Food and Drug Administration (FDA) blue book memorandum No. G95-1, entitled “Use of International Standard ISO-10993, Biological Evaluation of Medical Devices Part-1: Evaluation and Testing.” Typically, these tests measure a material's toxicity, infectivity, pyrogenicity, irritation potential, reactivity, hemolytic activity, carcinogenicity and/or immunogenicity. A biocompatible structure or material, when introduced into a majority of patients, will not cause a significantly adverse, long-lived or escalating biological reaction or response, and is distinguished from a mild, transient inflammation which typically accompanies surgery or implantation of foreign objects into a living organism.

The terms “biodegradable” and “bioerodible” refers to something, such graft material, implant, coating, or dressing, that when placed the in vivo environment of its intended use will eventually dissolute into constituent parts that may be metabolized or excreted, under the conditions normally present in a living tissue. In exemplary embodiments, the rate and/or extent of biodegradation or bioerosion may be controlled in a predictable manner.

“Therapeutic compound” or “therapeutic agent” means a compound or agent useful in the healing of damaged or diseased tissues on a patient's body.

The term “healing” means replacing, repairing, healing, or treating of damaged or diseased tissues of a patient's body.

The term “nucleic acid” refers to a polymeric form of nucleotides, either ribonucleotides or deoxynucleotides or a modified form of either type of nucleotide. The terms should also be understood to include, as equivalents, analogs of either RNA or DNA made from nucleotide analogs, and, as applicable to the embodiment being described, single-stranded (such as sense or antisense) and double-stranded polynucleotides.

The term “polypeptide”, and the terms “protein” and “peptide” which are used interchangeably herein, refers to a polymer of amino acids.

The term “therapeutically effective amount” refers to that amount of a modulator, drug or other molecule that is sufficient to effect treatment when administered to a subject in need of such treatment. The therapeutically effective amount will vary depending upon the subject and disease condition being treated, the weight and age of the subject, the severity of the disease condition, the manner of administration and the like, which can readily be determined by one of ordinary skill in the art.

As used herein, the term “tissue” refers to an aggregation of similarly specialized cells united in the performance of a particular function. Tissue is intended to encompass all types of biological tissue including both hard and soft tissue, including connective tissue (e.g., hard forms such as osseous tissue or bone) as well as other muscular or skeletal tissue. In a preferred embodiment tissue is skin.

The term “vector” refers to a nucleic acid capable of transporting another nucleic acid to which it has been linked. One type of vector which may be used herein is an episome, i.e., a nucleic acid capable of extra-chromosomal replication. Other vectors include those capable of autonomous replication and expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer to circular double stranded DNA molecules that, in their vector form are not bound to the chromosome. In the present specification, “plasmid” and “vector” are used interchangeably as the plasmid is the most commonly used form of vector. However, the present disclosure is intended to include such other forms of expression vectors which serve equivalent functions and which become known in the art subsequently hereto.

The term “condition” refers to any injury, disease, disorder or effect that produces deleterious biological consequences in a subject.

The terms “patient,” “subject,” and “recipient” as used in this application refer to any mammal, especially humans.

“Mammal” for purposes of treatment refers to any animal classified as a mammal, including humans, domestic and farm animals, and zoo, sports, or pet animals, such as dogs, horses, cats, cattle, pigs, sheep, etc. Preferably, the mammal is human.

### Extracellular Matrix (ECM)

In accordance with the present invention, the graft material includes an extracellular matrix and at least one therapeutic agent.

Upon application of the graft material to the body of the subject, ECM in the graft material may undergo remodeling and induce cell growth of endogenous tissues while delivering therapeutic agents. The ECM in the graft material may serve as a matrix for, promote and/or induce the growth of endogenous tissue and undergo a process of bioremodeling. Common events related to this bioremodeling process may include widespread and rapid neovascularization, proliferation of granulation mesenchymal cells, biodegradation/resorption of implanted purified intestine submucosa material, and lack of immune reaction. Therapeutic agents may advance the healing process by producing a desired biological effect in vivo (e.g., stimulation or suppression of cell division, migration or apoptosis; stimulation or suppression of an immune response; anti-bacterial activity; etc.).

Studies have shown that ECM materials such as warm-blooded vertebrate submucosa may be capable of inducing host tissue proliferation, bioremodeling and regeneration of tissue structures following implantation in a number of in vivo microenvironments including lower urinary tract, body wall, tendon, ligament, bone, cardiovascular tissues and the central nervous system. Upon implantation, cellular infiltration and a rapid neovascularization may be observed and the submucosa material may be bioremodeled into host replacement tissue with site-specific structural and functional properties. This may occur as a result of one or more of the components of submucosa including, for example, glycosaminoglycans, glycoproteins, proteoglycans, and/or growth factors, including Transforming Growth Factor-α, Transforming Growth Factor-β, and/or Fibroblast Growth Factor 2 (basic).

ECM is the noncellular part of a tissue and consists of protein and carbohydrate structures secreted by the resident cells. ECM serves as a structural element in tissues. The extracellular matrix can be isolated and treated in a variety of ways. When harvested from the tissue source and fabricated into a graft material, the ECMs may be referred to as naturally occurring polymeric scaffolds, bioscaffolds, biomatrices, ECM scaffolds, extracellular matrix material (ECMM), or naturally occurring biopolymers. The ECM materials, though harvested from several different body systems as described below, all share similarities when processed into a graft material. Specifically, since they are subjected to minimal processing after they are removed from the source animal, they retain a structure and composition nearly identical to their native state. The host cells are removed and the scaffolds may be implanted acellularly to replace or repair damaged tissues while delivering therapeutic agents to the tissue.

The ECM for use in preparing graft materials can be selected from a variety of commercially available matrices including collagen matrices, or can be prepared from a wide variety of natural sources of collagen. Examples of these naturally occurring ECMs include tela submucosa, acellular dermis, cadaveric fascia, the bladder acellular matrix graft, and amniotic membrane (for review see Hodde J., Tissue Engineering 8(2):295-308 (2002)). In addition, collagen-based extracellular matrices derived from renal capsules of warm blooded vertebrates may be selected for use in preparing the graft materials of this invention. The extracellular matrices derived from renal capsules of warm blooded vertebrates were described in WO 03/02165, the disclosure of which is incorporated herein by reference.

Another type of ECM, isolated from liver basement membrane, is described in U.S. Pat. No. 6,379,710, which is incorporated herein by reference. ECM may also be isolated from pericardium, as described in U.S. Pat. No. 4,502,159, which is also incorporated herein by reference.

In addition to xenogenic biomaterials, autologous tissue can be harvested as well. Additionally elastin or elastin-like polypeptides (ELPs) and the like offer potential as a biologically active ECM. Another alternative would be to use allographs such as harvested native valve tissue. Such tissue is commercially available in a cryopreserved state.

In one example, the ECM for use in accordance with the present invention comprises the collagenous matrix having highly conserved collagens, glycoproteins, proteoglycans, and glycosaminoglycans, and/or growth factors, including Transforming Growth Factor-α, Transforming Growth Factor-β, and/or Fibroblast Growth Factor 2 (basic), in their natural configuration and natural concentration. In another example, the collagenous matrix comprises submucosa-derived tissue of a warm-blooded vertebrate, such as small intestine submucosa (SIS). Submucosal tissue can be obtained from various vertebrate organ sources (such as intestinal tissue) harvested from animals raised for meat production, including, for example, pigs, cattle and sheep or other warm-blooded vertebrates.

Juvenile submucosa tissue from warm blooded vertebrates, such as a porcine mammal, may also be used. Juvenile submucosal tissue was described in WO 04/22107, the disclosure of which is incorporated herein by reference.

After the host cells are removed, the scaffolds may be implanted acellularly to replace or repair damaged tissues while, for example, delivering therapeutic agents to the tissue.

The ECM of the graft material may be, for example, tela submucosa. “Tela submucosa” or “submucosa” refers to a layer of collagen-containing connective tissue occurring under the mucosa in most parts of the alimentary, respiratory, urinary and genital tracts of animals. Tela submucosa is a preferred source of ECM. Purified tela submucosa, a preferred type of ECM, has been previously described in U.S. Pat. Nos. 6,206,931, 6,358,284 and 6,666,892 as a bio-compatible, non-thrombogenic material that enhances the repair of damaged or diseased host tissues. U.S. Pat. Nos. 6,206,931, 6,358,284 and 6,666,892 are incorporated herein by reference. The submucosa may be derived from intestine. The mucosa can also be derived from vertebrate liver tissue as described in WIPO Publication, WO 98/25637, based on PCT application PCT/US97/22727; from gastric mucosa as described in WIPO Publication, WO 98/26291, based on PCT application PCT/US97/22729; from stomach mucosa as described in WIPO Publication, WO 98/25636, based on PCT application PCT/US97/23010; or from urinary bladder mucosa as described in U.S. Pat. No. 5,554,389, the disclosures of all are expressly incorporated herein.

The submucosa is preferably derived from the intestines, more preferably the small intestine, of a warm blooded vertebrate; i.e., small intestine submucosa (SIS). SIS is commercially available from Cook Biotech, West Lafayette, Ind. Preferred intestine submucosal tissue typically includes the tunica submucosa delaminated from both the tunica muscularis and at least the luminal portions of the tunica mucosa. In one example the submucosal tissue includes the tunica submucosa and basilar portions of the tunica mucosa including the lamina muscularis mucosa and the stratum compactum. The preparation of intestinal submucosa is described in U.S. Pat. No. 4,902,508, and the preparation of tela submucosa is described in U.S. patent application Ser. No. 08/916,490, both of which are incorporated herein by reference. The preparation of submucosa is also described in U.S. Pat. No. 5,733,337 and in 17 Nature Biotechnology 1083 (November 1999); and WIPO Publication WO 98/22158, dated 28 May 1998, which is the published application of PCT/US97/14855. Also, a method for obtaining a highly pure, delaminated tela submucosa collagen matrix in a substantially sterile state was previously described in U.S. Patent Application, Publication No. 20040180042, disclosure of which is incorporated by reference.

The stripping of the tela submucosa source is preferably carried out by utilizing a disinfected or sterile casing machine, to produce a tela submucosa which is substantially sterile and which has been minimally processed. A suitable casing machine is the Model 3-U-400 Stridhs Universal Machine for Hog Casing, commercially available from the AB Stridhs Maskiner, Gotoborg, Sweden. As a result of this process, the measured bioburden levels may be minimal or substantially zero. Other means for delaminating the tela submucosa source can be employed, including, for example, delaminating by hand.

In this method, a segment of vertebrate intestine, preferably harvested from porcine, ovine or bovine species, may first be subjected to gentle abrasion using a longitudinal wiping motion to remove both the outer layers, identified as the tunica serosa and the tunica muscularis, and the innermost layer, i.e., the luminal portions of the tunica mucosa. The submucosal tissue is rinsed with water or saline, optionally sterilized, and can be stored in a hydrated or dehydrated state. Delamination of the tunica submucosa from both the tunica muscularis and at least the luminal portions of the tunica mucosa and rinsing of the submucosa provide an acellular matrix designated as submucosal tissue. The use and manipulation of such material for the formation of ligament and tendon grafts and the use more generally of such submucosal tissue constructs for inducing growth of endogenous connective tissues is described and claimed in U.S. Pat. No. 5,281,422 issued Jan. 25, 1994, the disclosure of which is incorporated herein by reference.

Following delamination, submucosa may be sterilized using any conventional sterilization technique including propylene oxide or ethylene oxide treatment and gas plasma sterilization. Sterilization techniques which do not adversely affect the mechanical strength, structure, and biotropic properties of the purified submucosa are preferred. Preferred sterilization techniques also include exposing the graft to ethylene oxide treatment or gas plasma sterilization. Typically, the purified submucosa is subjected to two or more sterilization processes. After the purified submucosa is sterilized, for example by chemical treatment, the matrix structure may be wrapped in a plastic or foil wrap and sterilized again using electron beam or gamma irradiation sterilization techniques.

Preferred submucosa may also be characterized by the low contaminant levels set forth in Table 1 below. The contaminant levels in Table 1 may be found individually or in any combination in a given ECM sample. The abbreviations in Table 1 are as follows: CFU/g=colony forming units per gram; PFU/g=plaque forming units per gram; μg/mg=micrograms per milligram; ppm/kg=parts per million per kilogram.

TABLE 1

| | | | Second | | Third | |
|---|---|---|---|---|---|---|
| | First Preferred | | Preferred | | Preferred | |
| | Level | | Level | | Level | |
| ENDOTOXIN | <12 | EU/g | <10 | EU/g | <5 | EU/g |
| BIOBURDEN | <2 | CFU/g | <1 | CFU/g | <0.5 | CFU/g |
| FUNGUS | <2 | CFU/g | <1 | CFU/g | <0.5 | CFU/g |
| NUCLEIC | <10 | μg/mg | <5 | μg/mg | <2 | μg/mg |
| ACID | | | | | | |
| VIRUS | <500 | PFU/g | <50 | PFU/g | <5 | PFU/g |
| PROCESSING | <100,000 | ppm/kg | <1,000 | ppm/kg | <100 | ppm/kg |
| AGENT | | | | | | |

Purified submucosa may be further processed in a number of ways to provide ECM suitable for incorporation into the graft material of this invention.

It is also known that comminuted forms of submucosa can be prepared without loss of the submucosal tissue's ability to induce the growth of endogenous tissues. Comminuted submucosa compositions are prepared as solutions or suspensions or powder of intestine submucosa and comprise mechanically obtained submucosa or enzymatically treated submucosa. In one example, the submucosal tissue is mechanically and enzymatically treated to form a substantially uniform or homogenous solution. In another example, the submucosa is treated with a protease, such as trypsin or pepsin, or other appropriate enzymes for a period of time sufficient to solubilize the tissue and form a substantially homogeneous solution.

Preferably, the intestine submucosa starting material is mechanically comminuted by tearing, cutting, grinding, shearing and the like. Grinding the submucosa in a frozen or freeze-dried state is preferred although good results can be obtained as well by subjecting a suspension of pieces of the submucosa to treatment in a high speed (high shear) blender and dewatering, if necessary, by centrifuging and decanting excess water. The resultant fluidized intestine submucosa can be dried to form a submucosa powder. Thereafter, it can be hydrated, that is, combined with water or buffered saline and optionally other pharmaceutically acceptable excipients to form a intestine submucosa composition as a fluid having a viscosity of about 2 to about 300,000 cps at 25° C. The higher viscosity submucosal compositions can have a gel or paste consistency. The fluidized compositions can be sterilized using art-recognized sterilization techniques such as exposure to ionizing radiation. The preparation of fluidized forms of intestine submucosa is described in U.S. Pat. Nos. 5,275,826, 5,516,533, and 6,264,992, the disclosures of which are incorporated herein by reference.

The intestine submucosa may also be in the form of powder of submucosal tissues. In one example a powder form of submucosal tissue is prepared by pulverizing intestine submucosa tissue under liquid nitrogen to produce particles ranging in size from 0.01 to 1 mm in their largest dimension. The particulate composition is then lyophilized overnight, pulverized again and optionally sterilized to form a substantially anhydrous particulate composite. In another example, a powder form of submucosal tissue can be formed from fluidized submucosal tissue by drying the suspensions or solutions of submucosal tissue.

Both solid and fluidized forms of intestine submucosa have been found to induce endogenous remodeling processes including rapid neovascularization, proliferation of granulation mesenchymal cells, resorption of the submucosa tissue and absence of immune rejection. In vivo, submucosa tissue has been found effective to induce the proliferation and growth of cells/tissues with which it is in contact or which it replaces.

It is also possible to form large surface area constructs by combining two or more tela submucosa sections using techniques described in U.S. Pat. Nos. 2,127,903 and 5,711,969, which are incorporated herein by reference. Thus, a plurality of tela submucosa strips can be fused to one another, for example by compressing overlapping areas of the strips under dehydrating conditions, to form an overall planar construct having a surface area greater than that of any one planar surface of the individual strips used to form the construct.

Variations of the above-described processing procedures may be used to produce submucosa that may be incorporated into a polymeric sheet of the graft material. For example, the source tissue for the tela submucosa, e.g., stomach, whole intestine, cow uterus and the like, can be partially delaminated, treated with a disinfecting or sterilizing agent followed by complete delamination of the tela submucosa. Illustratively, attached mesentery layers, and/or serosa layers of whole intestine can be removed prior to treatment with the disinfecting agent, followed by delamination of remaining attached tissues from the tela submucosa. These steps may or may not be followed by additional disinfection steps, e.g., enzymatic purification and/or nucleic acid removal. Alternatively, the tela submucosa source can be minimally treated with a disinfecting or other such agent, the tela submucosa delaminated from the tunica muscularis and tunica mucosa, followed by a complete disinfection treatment to attain the desired contaminant level(s). All such variations and modifications of this step are contemplated.

The purified submucosa can be conditioned, as described in U.S. patent application Ser. No. 08/916,490, to alter the viscoelastic properties of the purified submucosa. The purified submucosa may be conditioned by stretching, chemically treating, enzymatically treating or exposing the matrix structure to other environmental factors. In one embodiment, the strips of purified tela submucosa may be conditioned by stretching in a longitudinal and/or lateral direction to a strain of no more than 20%. Strain is the percentage increase in the length of the material after loading.

In another embodiment, the purified submucosa may be conditioned by stretching the material longitudinally to a length longer than the length of the purified submucosa from which the ECM was formed. One method of conditioning the matrix by stretching involves application of a given load to the purified submucosa for three to five cycles. Each cycle consists of applying a load to the material for five seconds, followed by a ten second relaxation phase. Three to five cycles produces a stretch-conditioned material. The purified submucosa does not immediately return to its original size; it remains in a “stretched” dimension. Optionally, the purified submucosa may be preconditioned by stretching in the lateral dimension.

In one embodiment the purified submucosa may be stretched using 50% of the predicted ultimate load. The “ultimate load” is the maximum load that can be applied to the purified submucosa without resulting in failure of the matrix structure (i.e., the break point of the tissue). Ultimate load can be predicted for a given strip of purified submucosa based on the source and thickness of the material. Accordingly, one method of conditioning the matrix structure by stretching involves application of 50% of the predicted ultimate load to the purified submucosa for three to ten cycles. Each cycle consists of applying a load to the material for five seconds, followed by a ten-second relaxation phase. The resulting conditioned purified submucosa has a resultant strain of less than 30%, more typically a strain from about 20% to about 28%. In one preferred embodiment, the conditioned purified submucosa has a strain of no more than 20%. The resultant conditioned purified submucosa can be used in the manner described below. The conditioning process and other relevant processes are described in U.S. Pat. No. 6,358,284 which is incorporated herein by reference.

The ECM of the graft material may be, for example, acellular dermis. Acellular dermis is composed of normal dermal tissue structures that remain after the cells are removed. Like other naturally occurring biopolymers, acellular dermis is rich in collagen type I. Acellular dermis also retains high levels of the type IV and type VII collagen composition of the native dermis (Medalie et al., ASAIO J. 42:M455 (1996)). In addition to collagen, the elastin content of the dermis is also retained during processing, leading to a graft construct with favorable elastic properties (Isch et al., J. Pediatr. Surg. 36:266 (2001)).

Acellular dermis may be harvested from either a pig or human cadaver skin. For example. Acellular dermis may be prepared according to Chaplin et al. (Chaplin et al., Neurosurgery 45:320 (1999)). Briefly, the epidermis may be removed by soaking the skin in sodium chloride (NaCl). Dermal fibroblasts and epithelial cells may be removed by incubation of the material in deoxycholic acid containing ethylenediaminetetraacetate (EDTA). The dermis may then be cryoprotected with a combination of maltodextrin and disodium-EDTA, and freeze dried until use (Chaplin et al., Neurosurgery 45:320 (1999)). When implanted as an acellular tissue graft, acellular dermis endothelializes repaired vascular structures (Inoue and Lleon, J. Reconstr. Microsurg. 12:307 (1996)), inhibits excessive wound contraction (Walden et al., Ann. Plast. Surg. 45:162 (2000)), and supports host cell incorporation and capillary ingrowth into the grafted site (Dalla et al., J. Pediatr. Surg. 45:162 (2000); and Medalie et al., ASAIO J. 42:M455 (1996)).

The ECM of the graft material may be, for example, cadaveric fascia. The tensor fascia lata is thick band of connective tissue attaching the pelvis to the knee on the lateral side of the leg. Its muscular components at the hip join to thick connective tissues that help stabilize and steady the hip and knee joints by putting tension on the iliotibial band (IT band). The IT band, the distal section of the tensor fascia lata, may be harvested for the ECM of the graft material of this invention.

In its native state, the fascia lata tendon is composed of heavy, parallel bundles of type I collagenous fibers that are held together by extracellular matrix tissue. Between the bundles of fibers are fibroblasts, nerves, and blood vessels that supply the tendon with nutrients. Cadaveric fascia may be obtained by ethanol extraction followed by high-pressure washing with antibiotics. The extracted tissue may then be lyophilized and terminally sterilized with gamma irradiation. Intraoperatively, the graft material may be reconstituted with saline soak prior to use (Carbone et al., J. Urol. 165:1605 (2001)).

The ECM of the graft material may be, for example, bladder acellular matrix. Bladder acellular matrix graft (BAMG) was first described in 1975 (Meezan et al, Life Sci. 17:1721 (1975)) and may be derived from a layer of the urinary bladder that is analogous to the submucosal tissue comprising the bulk of SIS biomaterial. In the native bladder, the bladder submucosa supports the mucosal structures and is secreted and maintained by fibroblasts. The normal function of ECM is to support the growth and differentiation of different mucosal cell types while maintaining a connective tissue structure that gives integrity to the bladder wall. Unlike the intestinal submucosa, however, which is easily separated from the external muscle layers, the submucosa of the urinary bladder is intimately attached to the muscular bladder wall. Complete mechanical separation of the layers have proven tedious and difficult, and so attempts at rendering the bladder submucosa muscle-free have often resorted to chemical and/or enzymatic agents such as sodium hydroxide, sodium desoxycholate, sodium dodecyl sulfate (SDS), or deoxyribonuclease (Badylak et al., J. Pediatr. Surg. 35:1097 (2000); Merguerian et al., BJU Int. 85:894 (2000); Wefer et al., J. Urol. 165:1755 (2001); and Reddy et al., J. Urol. 164:936 (2000)).

In one processing method, whole bladders may be soaked in a Tris-EDTA solution for 48 hours followed by additional soaking in Tris-potassium chloride-EDTA solution containing Triton-X. Bladders may then be rinsed in Sorenson's phosphate buffer solution, incubated overnight with deoxyribonuclease and ribonuclease to remove cytoplasmic and nuclear material, and further extracted in a solution containing Tris and SDS. The extracted bladders may then be submerged in ethanol to remove any residual SDS, washed in phosphate buffer, and stored in refrigerated saline until use (Reddy et al., J. Urol. 164:936 (2000)).

Alternatively, bladder submucosa may be rendered acellular and sterile according to the methods used for SIS (Badylak et al., J. Pediatr. Surg. 35:1097 (2000)). The bladder layers may be mechanically separated and the resulting submucosa thoroughly rinsed in water to lyse the cells. The submucosa may be treated with peracetic acid and then rinsed in sequential exchanges of water and phosphate buffered saline to yield a neutral pH. It may then be sterilized using 2.5-mRad gamma irradiation and stored refrigerated until use.

The ECM of the graft material may be, for example, amniotic membrane. The amniotic membrane forms the sac that encloses the embryo during pregnancy. It is extremely strong, 2-5 μg-thick tissue that may be used as a graft material in several tissue repair applications. In its native state, the epithelium of the amnion consists of a single layer of cells resting upon a relatively cell-free basement membrane ECM (Aplin et al., J. Cell Sci. 79:119 (1985)). This ECM consists of a microscopic substructure consisting of lamina rara and lamina densa that is comprised of several collagen types, including the fibrillar collagen types I and III, and the basal lamina collagen type IV (Aplin et al., J. Cell Sci. 79:119 (1985); and Lei et al., Biol. Reprod. 60:176 (1999)). At least one proteoglycan, decorin, has been identifies in near-term amniotic membrane (Meinert et al., J. Obstet. Gynecol. 184:679 (2001)), and has the glycosaminoglycan, hyaluronic acid (Meinert et al., J. Obstet. Gynecol. 184:679 (2001)). Several growth factors, including epidermal growth factor, several transforming growth factor isoforms, basic fibroblast growth factor, keratinocyte growth factor, and hepatocyte growth factor also have been identified and have been reported to be retained in the processed tissue matrix.

Amniotic membrane may be obtained at parturition and cleaned of blood with saline containing penicillin, streptomycin, amphotericin B, and clindamycin (Avila et al., Cornea 20:414 (2001). It may be separated from chorion by blunt dissection, washed in sterile water, and treated by soaking for 3 hours in a 10% solution of trypsin to lyse the cells. The membrane may then be sterilized with gamma irradiation and frozen until clinical use (Young et al., Fertil. Steril. 55:624 (1991)).

### Therapeutic Agents

In accordance with the present invention, the graft material also contains at least one therapeutic agent.

Therapeutic agents present in the graft material as previously mentioned, are capable of producing a desired biological effect in vivo (e.g., stimulation or suppression of cell division, migration or apoptosis; stimulation or suppression of an immune response; anti-bacterial activity; etc.).

Suitable therapeutic agents include growth factors, antibiotics, anti-viral agents, analgesics, anti-inflammatories, both steroidal and non-steroidal, anti-neoplastics, anti-spasmodics including channel blockers, modulators of cell-extracellular matrix interactions including cell growth inhibitors and anti-adhesion molecules, enzymes and enzyme inhibitors, anticoagulants and/or antithrombotic agents, DNA, RNA, inhibitors of DNA, RNA or protein synthesis, compounds modulating cell migration, proliferation and/or growth, vasodilating agents, and other drugs commonly used for the treatment of injury to tissue.

Combinations of these therapeutic agents may also be used.

Therapeutic agents may be, for example, substances that enhance or exclude particular varieties of cellular or tissue ingrowth. Such substances include, for example, osteoinductive, angiogenic, mitogenic, or similar substances, such as transforming growth factors (TGFs), for example, TGF-alpha, TGF-beta-1, TGF-beta-2, TGF-beta-3; fibroblast growth factors (FGFs), for example, acidic and basic fibroblast growth factors (aFGF and bFGF); platelet derived growth factors (PDGFs); platelet-derived endothelial cell growth factor (PD-ECGF); tumor necrosis factor alpha (TNF-alpha); tumor necrosis factor beta (TNF-b); epidermal growth factors (EGFs); connective tissue activated peptides (CTAPs); osteogenic factors, for example, for example, BMP-1, BMP-2, BMP-3 MP-4, BMP-5, BMP-6, BMP-7, BMP-8, BMP-9; insulin-like growth factor (IGF), for example, IGF-I and IGF-II; erythropoietin; heparin binding growth factor (hbgf); vascular endothelium growth factor (VEGF); hepatocyte growth factor (HGF); colony stimulating factor (CSF); macrophage-CSF (M-CSF); granulocyte/macrophage CSF (GM-CSF); nitric oxide synthase (NOS); nerve growth factor (NGF); muscle morphogenic factor (MMP); Inhibins (for example, Inhibin A, Inhibin B); growth differentiating factors (for example, GDF-1); Activins (for example, Activin A, Activin B, Activin AB); angiogenin; angiotensin; angiopoietin; angiotropin; antiangiogenic antithrombin (aaAT); atrial natriuretic factor (ANF); betacellulin; endostatin; endothelial cell-derived growth factor (ECDGF); endothelial cell growth factor (ECGF); endothelial cell growth inhibitor; endothelial monocyte activating polypeptide (EMAP); endothelial cell-viability maintaining factor; endothelin (ET); endothelioma derived mobility factor (EDMF); heart derived inhibitor of vascular cell proliferation; hematopoietic growth factors; erythropoietin (Epo); interferon (IFN); interleukins (IL); oncostatin M; placental growth factor (PlGF); somatostatin; transferring; thrombospondin; vasoactive intestinal peptide; and biologically active analogs, fragments, and derivatives of such growth factors.

In exemplary embodiments, the therapeutic agents are growth factors, angiogenic factors, compounds selectively inhibiting ingrowth of fibroblast tissue such as anti-inflammatories, and compounds selectively inhibiting growth and proliferation of transformed (cancerous) cells. These factors may be utilized to control the growth and function of cells contained within or surrounding the ECM of the graft material, including, for example, the ingrowth of blood and/or the deposition and organization of fibrous tissue around the graft material.

Therapeutic agents may be, for example, polynucleotides. Examples of polynucleotides which are useful as therapeutic agents include, but are not limited to, nucleic acids and fragments of nucleic acids, including, for example, DNA, RNA, cDNA and recombinant nucleic acids; naked DNA, cDNA, and RNA; genomic DNA, cDNA or RNA; oligonucleotides; aptomeric oligonucleotides; ribozymes; anti-sense oligonucleotides (including RNA or DNA); DNA coding for an anti-sense RNA; DNA coding for tRNA or rRNA molecules (i.e., to replace defective or deficient endogenous molecules); double stranded small interfering RNAs (siRNAs); polynucleotide peptide bonded oligos (PNAs); circular or linear RNA; circular single-stranded DNA; self-replicating RNAs; mRNA transcripts; catalytic RNAs, including, for example, hammerheads, hairpins, hepatitis delta virus, and group I introns which may specifically target and/or cleave specific RNA sequences in vivo; polynucleotides coding for therapeutic proteins or polypeptides, as further defined herein; chimeric nucleic acids, including, for example, DNA/DNA hybrids, RNA/RNA hybrids, DNA/RNA hybrids, DNA/peptide hybrids, and RNA/peptide hybrids; DNA compacting agents; and gene/vector systems (i.e., any vehicle that allows for the uptake and expression of nucleic acids), including nucleic acids in a non-infectious vector (i.e., a plasmid) and nucleic acids in a viral vector.

In an exemplary embodiment, chimeric nucleic acids include, for example, nucleic acids attached to a peptide targeting sequences that directs the location of the chimeric molecule to a location within a body, within a cell, or across a cellular membrane (i.e., a membrane translocating sequence (“MTS”)).

In another embodiment, a nucleic acid may be fused to a constitutive housekeeping gene, or a fragment thereof, which is expressed in a wide variety of cell types.

In certain embodiments, polynucleotides delivered by non-viral methods may be formulated or associated with nanocaps (e.g., nanoparticulate CaPO₄), colloidal gold, nanoparticulate synthetic polymers, and/or liposomes. In one embodiment, polynucleotides may be associated with QDOT™ Probes (www.qdots.com).

In other embodiments, polynucleotides useful as therapeutic agents may be modified so as to increase resistance to nucleases, e.g. exonucleases and/or endonucleases, and therefore have increased stability in vivo. Exemplary modifications include, but are not limited to, phosphoramidate, phosphothioate and methylphosphonate analogs of nucleic acids (see also U.S. Pat. Nos. 5,176,996; 5,264,564; and 5,256,775).

In certain embodiments, the therapeutic agent is a polynucleotide that is contained within a vector. Suitable vectors for use in accordance with the present invention include, for example, viral vectors or vectors derived from viral sources, such as adenoviral vectors, herpes simplex vectors, papilloma vectors, adeno-associated vectors, retroviral vectors, pseudorabies virus, alpha-herpes virus vectors, and the like. A thorough review of viral vectors, particularly viral vectors suitable for modifying nonreplicating cells, and how to use such vectors in conjunction with the expression of polynucleotides of interest can be found in the book Viral Vectors: Gene Therapy and Neuroscience Applications Ed. Caplitt and Loewy, Academic Press, San Diego (1995).

Vectors may be, for example, non-infectious vectors, or plasmids. Suitable non-infectious vectors, include, but are not limited to, mammalian expression vectors that contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, PMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. The various methods employed in the preparation of the plasmids and transformation of host organisms are well known in the art. For other suitable expression systems for both prokaryotic and eukaryotic cells, as well as general recombinant procedures, see Molecular Cloning A Laboratory Manual, 2nd Ed., ed. by Sambrook, Fritsch and Maniatis (Cold Spring Harbor Laboratory Press, 1989) Chapters 16 and 17.

Therapeutic agents may be, for example, inhibitors of DNA, RNA, or protein synthesis.

Therapeutic agents may be, for example, other biologically active molecules that exert biological effects in vivo. These therapeutic agents used in conjunction with the ECM as the graft material of this invention, include, antibiotics, anti-fungal agents, antiviral agents, analgesics, anti-inflammatories, both steroidal and non-steroidal, anti-neoplastics, anti-spasmodics including channel blockers, modulators of cell-extracellular matrix interactions including cell growth inhibitors and anti-adhesion molecules, enzymes and enzyme inhibitors (angiotensin converting enzyme inhibitor compound), anticoagulants and/or antithrombotic agents, inhibitors of DNA, RNA or protein synthesis, compounds modulating cell migration, proliferation and/or growth, vasodilating agents, and other drugs commonly used for the treatment of injury to tissue. For examples and detailed description of these therapeutic agents see Goodman & Gilman's The Pharmacological Basis of Therapeutics, 9^{th} Edition.

Specific examples of antibiotics include, but are not limited to, penicillins, including aminopenicillins (Ampicillin, Amoxicillin, and their congeners); cephalosporins; cycloserine; vancomycin; polymyxin; amphotericin B; chloramphenicol; tetracyclines (Chlortetracycline, Oxytetracycline, Demeclocycline, Methacycline, Doxycycline, and Minocycline); macrolides (Erythromycin, Clarithromycin, Azithromycin); clindamycin; rifamycins; quinolones; sulfonamides; rifamycins, including rifampin (RIFADIN; RIMACTANE); ethambuto; and other available antibiotic agents.

For example, silver sulfadiazine (brand names: Silvadene, SSD, SSD AF, Thermazene), a sulfa drug, may be used to prevent and treat bacterial or fungus infections.

Specific examples of systemic and topical anti-fungal agents include amphotericin B, flucytozine, imidazoles and triazoles, ketoconazole, itraconazole, fluconazole, ciclopirox olamine, haloprogin, tolnaftate, naftifine, terbinafine, and polyene antifungal antibiotics (nystatin).

Specific examples of antiviral agents include, but are not limited to, antiretroviral agents (didanosine, stavudine, zalcidabine, zidovudine), antiherpesvirus agents (acyclovir, famciclovir, foscarnet, trifluridine, vidarabile), and other antiviral agents (amantadine, interferon alpha, ribavirin, rimantadine).

Specific examples of non-steroidal anti-inflammatory agents include, but are not limited to, salicylic acid derivatives (aspirin, sodium salicylate), para-aminophenol derivatives (acetaminophen), indole and indene acetic acids (indomethacin), heteroaryl acetic acids (tolmetin), arylpropionic acids (ibuprofen, naproxen, ketoprofen), anthanilic acids (mefenamic acid), enolic acids (piroxicam, phenylbutazone), and alkanones (nabumetone).

Specific examples of anti-neoplastics include, but are not limited to, alkylating agents (nitrogen mustards, triazenes), antimetabolites (folic acid analogs, pyrimidine analogs), natural products (antibiotics, enzymes), hormones and antagonists (progestins, estrogens, androgens), and other miscellaneous agents (adenocortical suppressant, substituted urea).

Specific examples of inhibitors of platelet aggregation, i.e. anticoagulant compounds and/or anti-thrombotic agents, include prostacyclin, heparin, streptokinase, urokinase, tissue plasminogen activator (TPA) and anisoylated plasminogen activator (TPA) and anisoylated plasminogen-streptokinase activator complex (APSAC).

Exemplary clot dissolving agents are tissue plasminogen activator, streptokinase, urokinase, and heparin.

Specific examples of channel blockers include calcium channel blocking drugs.

Specific examples of modulators of cell interactions include interleukins, platelet derived growth factor, acidic and basic fibroblast growth factor (FGF), transformation growth factor β (TGF-beta), epidermal growth factor (EGF), insulin-like growth factor, and antibodies thereto.

Specific examples of nucleic acids include genes and cDNAs encoding proteins, expression vectors, antisense and other oligonucleotides such as ribozymes which can be used to regulate or prevent gene expression.

Specific examples of other bioactive agents include modified extracellular matrix components or their receptors, and lipid and cholesterol sequestrants.

In certain embodiments, therapeutic agents may be pharmaceutical compositions or drugs, including small organic molecules, including, for example, antibiotics and anti-inflammatories.

Therapeutic agent may be, for example, used in conjunction with a coating to include proteins, such as cytokines, interferons and interleukins, poietins, and colony-stimulating factors. Carbohydrates including Sialyl Lewis which has been shown to bind to receptors for selectins to inhibit inflammation.

A ‘Deliverable growth factor equivalent’ (abbreviated DGFE), a growth factor for a cell or tissue, may be used, which is broadly construed as including growth factors, cytokines, interferons, interleukins, proteins, colony-stimulating factors, gibberellins, auxins, and vitamins; further including peptide fragments or other active fragments of the above; and further including vectors, i.e., nucleic acid constructs capable of synthesizing such factors in the target cells, whether by transformation or transient expression; and further including effectors which stimulate or depress the synthesis of such factors in the tissue, including natural signal molecules, antisense and triplex nucleic acids, and the like. Exemplary DGFE's are VEGFs, ECGF, bFGF, BMP, and PDGF, and DNA's encoding for them.

Therapeutic agents may be, for example, drugs having antioxidant activity (i.e., destroying or preventing formation of active oxygen), which are useful, for example, in the prevention of adhesions. Examples include superoxide dismutase, or other protein drugs include catalases, peroxidases and general oxidases or oxidative enzymes, such as cytochrome P450, glutathione peroxidase, and other native or denatured hemoproteins.

Therapeutic agents may be, for example, analgesic agents. Analgesic agents may be used for pain relief or pain suppression, especially for treatment of burns. Examples of the analgesic agents include, but are not limited to, previously mentioned nonsteroidal anti-inflammatory drugs, and opioids, such as morphine, methadone, codeine, etorphine, naloxone, and others.

Therapeutic agents may be, for example, mammalian stress response proteins or heat shock proteins, such as heat shock protein 70 (hsp 70) and hsp 90, or those stimuli which act to inhibit or reduce stress response proteins or heat shock protein expression, for example, flavonoids.

Therapeutic agents (i.e., polypeptides, polynucleotides, small molecules, drugs, etc.), for example, may be mixed with or encapsulated in a substance that facilitates its delivery to and/or uptake by cells in tissues.

In one embodiment, polynucleotides may be mixed with cationic lipids that are useful for the introduction of nucleic acid into the cell, including, but not limited to, LIPOFECTIN™ (DOTMA) which consists of a monocationic choline head group that is attached to diacylglycerol (see generally, U.S. Pat. No. 5,208,036 to Epstein et al.); TRANSFECTAM™ (DOGS) a synthetic cationic lipid with lipospermine head groups (Promega, Madison, Wis.); DMRIE and DMRIE.HP (Vical, La Jolla, Calif.); DOTAP™ (Boehringer Mannheim (Indianapolis, Ind.), and Lipofectamine (DOSPA) (Life Technology, Inc., Gaithersburg, Md.).

In other embodiments, therapeutic agents (i.e., polypeptides, polynucleotides, small molecules, drugs, etc.) may be mixed with or encapsulated into microspheres or nanospheres that promote penetration into mammalian tissues and uptake by mammalian cells. In various embodiments, the microspheres or nanospheres may optionally have other molecules bound to them. These modifications may, for example, impart the microspheres or nanospheres with the ability to target and bind specific tissues or cells, allow them be retained at the administration site, protect incorporated bioactive agents, exhibit antithrombogenic effects, prevent aggregation, and/or alter the release properties of the microspheres. Production of such surface-modified microspheres is discussed in Levy et al., PCT Application No. WO 96/20698, the disclosure of which is hereby incorporated by reference.

In exemplary embodiments, it may be desirable to incorporate receptor-specific molecules into or onto the microspheres to mediate receptor-specific particle uptake, including, for example, antibodies such as IgM, IgG, IgA, IgD, and the like, or any portions or subsets thereof, cell factors, cell surface receptors, MHC or HLA markers, viral envelope proteins, peptides or small organic ligands, derivatives thereof, and the like.

Therapeutic agents (i.e., polypeptides, polynucleotides, small molecules, drugs, cells, etc.), for example, may be mixed or complexed with particulates that promote delivery to, or uptake by mammalian cells, provide osteoconductive properties, influence mass transport, etc. Suitable particulates include bioceramics such as hydroxyapatite (“HA”) or other calcium containing compounds such as mono-, di-, octa-, alpha-tri-, beta-tri-, or tetra-calcium phosphate, fluoroapatite, calcium sulfate, calcium fluoride and mixtures thereof; bioactive glass comprising metal oxides such as calcium oxide, silicon dioxide, sodium oxide, phosphorus pentoxide, and mixtures thereof; and the like. In an exemplary embodiment, hydroxyapatite is used as the bioceramic material because it provides osteoinductive and/or osteoconductive properties. It is preferable that the particle size of the particulates be about 0.1 nm to about 100 nm, more preferably about 2 nm to about 50 nm.

Therapeutic agents may be formulated, for example, so as to provide controlled release over time, for example, days, weeks, months or years, as the ECM is degraded or eroded. In an exemplary embodiment, degradation of the ECM is modulated by an agent that decreases (e.g., via a peptide, protein, or chemical protease, such as, for example, aprotinin) or increases (e.g., a protease) the rate of degradation and/or erosion of the ECM. Alternatively, the therapeutic agents may comprise a microsphere composition which is attached to or incorporated within the ECM. In this embodiment, the ECM need not degrade in order to produce a time released effect of the therapeutic agents. Release properties can also be determined by the size and physical characteristics of the microspheres.

Therapeutic agents may also include, for example, adjuvants and additives, such as stabilizers, fillers, antioxidants, catalysts, plasticizers, pigments, and lubricants, to the extent such ingredients do not diminish the utility of the therapeutic agent for its intended purpose.

### Preparation of the Graft Materials

Graft materials of this invention are prepared with therapeutic agents to provide delivery of a therapeutic agent at a site of injury.

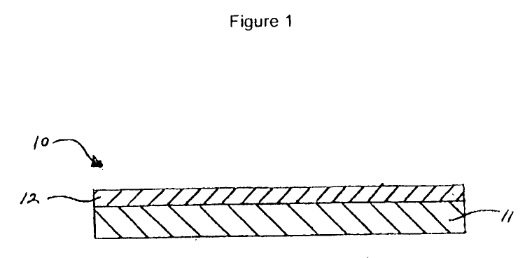

Therapeutic agents, for example, may be incorporated into the ECM or covalently attached to the ECM during the process of preparing of the graft material. Alternatively, therapeutic agents may be added to the ECM after preparation of the ECM, e.g., by soaking, spraying, painting, or otherwise applying the therapeutic agent to the ECM. For example, FIG. 1 is a schematic illustration wherein the graft material 10 comprises ECM 11. Therapeutic agents 12 may be applied by spraying one side of the ECM 11.

In various embodiments, therapeutic agents may be applied to the ECM directly at a desired location or may be pre-applied before application to the patient.

Graft materials may be in the form of flat films that may be adhered to tissue to cover the site of an injury or may be in the form of 3-D structures such as plugs or wedges. In another example, the graft material may be in a form of solid sheet, strip, gel, or powder.

Graft materials may be supplied in standard configurations suitable for application to a variety of wounds and may be applied as is or may be cut, molded or otherwise shaped prior to application to a particular application site. Alternatively, graft materials may be produced in a configuration tailored to a specific injury, disease, scar, wound or wound type.

Graft materials may be used for localized applications. Alternatively, whole graft materials may be used.

In one embodiment, the graft material is supplied as a moist material that is ready for application to a site on a patient's body. In another embodiment, the graft material is supplied as a dried material which may be rehydrated upon or prior to application to a body.

In yet another embodiment of this invention, therapeutic agents may be mixed with the fluidized ECM, such as fluidized SIS to form a substantially homogenous graft material solution including the ECM and desired therapeutic agents. In this case, the fluidized graft material is then applied to a patient's body.

In yet another embodiment of this invention, therapeutic agents may be first mixed with the fluidized ECM, such as fluidized SIS to form a substantially homogenous graft material including the ECM and desired therapeutic agents. The fluidized graft material is then allowed to dry before applying it to a patient. A method of preparing a fluidized or comminuted small intestine submucosa is described in Example 1 below.

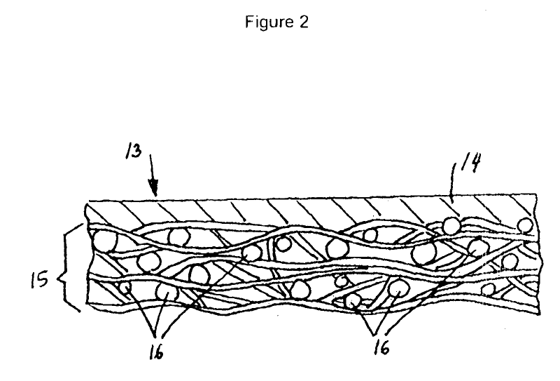

FIG. 2 shows a schematic illustration of a graft material 13 that comprises ECM 15 and wherein the therapeutic agents 16 are incorporated into the ECM 15 by mixing the therapeutic agents with a fluidized ECM and allowing the graft material to dry. Therapeutic agents also form a layer 14 on the surface of the ECM.

In certain embodiments, graft materials are prepared with therapeutic agents to provide delivery of a therapeutic agent at a desired location. Therapeutic agents may be included in a coating as an ancillary to a medical treatment (for example, antibiotics) or as the primary objective of a treatment (for example, a gene to be locally delivered). A variety of therapeutic agents may be used, including passively functioning materials such as hyaluronic acid, as well as active agents such as growth hormones. Specific examples of therapeutic agents of the graft materials of this invention were discussed previously.

### Therapy

The methods of the present invention are useful for healing of damaged or diseased tissues on a patient's body.

The graft materials formed and used in accordance with the present invention, upon placement on the damaged or diseased tissue on a patient's body, serve as rapidly vasularized matrix for support and growth of new endogenous tissue while delivering the therapeutic agents to the injured or diseases parts of patient's body in need of such treatment. The graft material may be then remodeled (resorbed and replaced with autogenous differentiated tissue) and assumes the characterizing features of the tissue with which the graft material is associated at the site of placement.

For example, because of the advantageous properties of the graft materials of this invention, the necessity for repeated debridement of a part of a patient's body in need of the treatment with the graft material may be reduced.

In one embodiment, the present invention encompasses a method for promoting healing of tissues. The method comprises contacting a tissue in need of healing with a graft material comprising an ECM and at least one therapeutic agent.

Therapeutic agents often have a specified function. For example, a therapeutic agent present in the graft material of this invention may be in an amount effective to promote endogeneous tissue growth at the site the graft material is placed. A therapeutically effective amount of therapeutic agents present in the graft material is expected to vary from about 0.1 milligram per kilogram of body weight per day (mg/kg/day) to about 100 mg/kg/day.

Those skilled in the art of treating damaged or diseased tissue in humans will know the dosages of the therapeutic agents for incorporation into the graft material to treat humans. In general, the effective therapeutic amount is adjusted for body surface area requiring such treatment.

Determination of therapeutically effective amounts of therapeutic agents of this invention may be readily made by the physician or veterinarian (the “attending clinician”). The dosages may be varied depending upon the requirements of the patient, the severity of the condition being treated and the particular agent being employed. In determining the dose, a number of factors are considered by the attending clinician, including, but not limited to: the specific tissue to be treated; pharmacodynamic characteristics of the particular agent; the desired time course of treatment; the species of mammal; its size, age, and general health; the specific disease involved; the degree of or involvement or the severity of the disease; the response of the individual patient; the particular compound administered; the mode of administration; the bioavailability characteristics of the preparation administered; the dose regimen selected; the kind of concurrent treatment; and other relevant circumstances.

In one embodiment, damaged or diseased portions of the patient's body may be repaired by placing a patch of a graft material including the ECM matrix and at least one therapeutic agent.

In another embodiment, the graft material disclosed herein may be used to create bioresorbable wound dressings or band-aids. Wound dressings may be used as a wound-healing dressing, a tissue sealant (i.e., sealing a tissue or organ to prevent exposure to a fluid or gas, such as blood, urine, air, etc., from or into a tissue or organ), and/or a cell-growth scaffold. In various embodiments, the wound dressing may protect the injured tissue, maintain a moist environment, be water permeable, be easy to apply, not require frequent changes, be non-toxic, be non-antigenic, maintain microbial control, and/or deliver effective healing agents to the wound site.

Examples of bioresorbable sealants and adhesives that may be used in accordance with the graft material described herein include, for example, FOCALSEAL® (biodegradable eosin-PEG-lactide hydrogel requiring photopolymerization with Xenon light wand) produced by Focal; BERIPLAST® produced by Adventis-Bering; VIVOSTAT® produced by ConvaTec (Bristol-Meyers-Squibb); SEALAGEN™ produced by Baxter; FIBRX® (containing virally inactivated human fibrinogen and inhibited-human thrombin) produced by CyoLife; TISSEEL® (fibrin glue composed of plasma derivatives from the last stages in the natural coagulation pathway where soluble fibrinogen is converted into a solid fibrin) and TISSUCOL® produced by Baxter; QUIXIL® (Biological Active Component and Thrombin) produced by Omrix Biopharm; a PEG-collagen conjugate produced by Cohesion (Collagen); HYSTOACRYL® BLUE (ENBUCRILATE) (cyanoacrylate) produced by Davis & Geck; NEXACRYL™ (N-butyl cyanoacrylate), NEXABOND™, NEXABOND™ S/C, and TRAUMASEAL™ (product based on cyanoacrylate) produced by Closure Medical (TriPoint Medical); DERMABOND™ which consists of 2-Octyl Cyanoacrylate produced by Dermabond (Ethicon); TISSUEGLU® produced by Medi-West Pharma; and VETBOND™ which consists of n-butyl cyanoacrylate produced by 3M.

Wound dressings may be used for soft tissue repair, including nerve repair, organ repair, skin repair, vascular repair, muscle repair, and ophthalmic applications. In exemplary embodiments, wound dressings may be used to treat a surface such as, for example, a surface of the dermis and epidermis, the site of an anastomosis, a suture, a staple, a puncture, an incision, a laceration, or an apposition of tissue.

In exemplary embodiments, wound dressings may be used in association with any medical condition that requires coating or sealing of a tissue. For example, bodily fluids may be stopped or minimized; barriers may be applied to prevent post-surgical adhesions, including those of the pelvis and abdomen, pericardium, spinal cord and dura, tendon and tendon sheath. Wound dressings may also be useful for treating exposed skin, in the repair or healing of incisions, abrasions, burns, inflammation, and other conditions requiring application of a coating to the outer surfaces of the body. Preferably, the graft material of this invention is used to treat skin.

In one example, burns may be treated with the graft material of this invention, wherein the graft material includes ECM and therapeutic agent such as silver sulfadiazine, antibiotics, or pain reliving agents and or a combination of these agents.

In each case, appropriate therapeutic agents are included in the graft material of this invention used as wound dressing to repair, replace, or heal damaged or diseased tissue on a patient's body.

This invention is further illustrated by the following experimental examples, which should not be construed as limiting. The contents of all references, patents and published applications cited throughout this application are hereby incorporated by reference herein.

### EXAMPLES

### Example 1

### Method of Preparing Fluidized Graft Material

The fluidized graft material may be prepared as a solution or suspension of intestinal submucosa. The intestinal submucosa starting material is comminuted by tearing, cutting, grinding, shearing and the like or may be digested with a protease, such as trypsin or pepsin, for a period of time sufficient to solubilize the tissue and form a substantially homogenous solution of submucosa.

The specimens are placed in a flat bottom stainless steel container and liquid nitrogen is introduced into the container to freeze the specimens to prepare them for further comminuting.

The frozen submucosal specimens are then comminuted to form coarse submucosal powder. Such processing may be carried out, for example with a manual arbor press with a cylindrical brass ingot placed on top of the frozen specimens. The ingot serves as an interface between the specimens and the arbor of the press. It is typically necessary to add liquid nitrogen periodically to the submucosal specimens to keep them frozen.

Alternatively, the suspension of pieces of submucosa may be subjected to the treatment in a high speed (high shear) blender and dewatering, if necessary by centrifugation and decanting excess water. A submucosal powder is produced. Thereafter, the submucosal powder may be re-hydrated using, for example buffered saline combined with therapeutic agents to form a fluidized tissue graft material at desired viscosity, for example viscosity of about 2 to about 300,000 cps at 25° C.

The higher viscosity graft materials may have a gel or paste consistency.

The graft material is then sterilized using art-recognized sterilization techniques such as exposure to ionizing radiation.

### Example 2

### Method for Treating a Wound

A graft material including SIS and antibiotics is used to treat a full-thickness skin wound. The graft material is a sheet of SIS wherein the antibiotics are applied by spraying the graft material on one side.

Skin wounds including second degree burns, lacerations, tears and abrasions; surgical excision wounds from removal of cancerous growth or autograft skin donor sites; and skin ulcers such as venous, diabetic, pressure (bed sores), and other chronic ulcers are managed using graft material comprising SIS and therapeutic agents.

Before the graft material is applied to the wound, the wound bed is prepared for its application.

Patients with burn wounds requiring grafting are selected. Graft material is placed directly on the excised wound bed with the side including antibiotics facing the wound.

The burned wounds sites to be grafted are prepared, such as by debridement, prior treatment according to standard practice so that the burned skin area was completely excised. Excised beds appear clean and clinically uninfected.

Patients undergoing surgical excision are locally anesthetized. The pre-operative area is cleansed with an anti-microbial/antiseptic skin cleanser (Hibiclens®) and rinsed with normal saline. Deep partial thickness wounds are made in the skin and the skin is grafted elsewhere unless it is cancerous. Graft material is applied to the wound bed and sterile bandages are applied.

In either wound case appropriate wound care is provided to the patient in examination, cleaning, changing bandages, etc. of the treated wounds.

Treatment of the wounds with the graft material of this invention may reduce the necessity for repeated debridement.

A complete record if the condition of the treated sites is maintained to document all procedures, necessary medications, frequency of dressing changes and any observations made. The wound beds remain protected from the external environment and moist to aid in wound management and healing.

It is therefore intended that the foregoing detailed description be regarded as illustrative rather than limiting, and that it be understood that it is the following claims, including all equivalents, that are intended to define the spirit and scope of this invention.

1. A graft material comprising:
an extracellular matrix (ECM); and
at least one therapeutic agent.

2. The graft material of claim 1, wherein the ECM is an extracellular collagenous matrix.

3. The graft material of claim 2, wherein the extracellular collagenous matrix comprises collagens, glycoproteins, proteoglycans, and glycosaminoglycans.

4. The graft material of claim 1, wherein the ECM is selected from the group consisting of small intestine submucosa, acellular dermis, cadaveric fascia, the bladder acellular matrix, and amniotic membrane.

5. The graft material of claim 4, wherein the ECM is the small intestine submucosa.

6. The graft material of claim 5, wherein the small intestine submucosa is fluidized.

7. The graft material of claim 1, wherein the therapeutic agent is selected from the group consisting of growth factors, antibiotics, anti-viral agents, analgesics, steroidal anti-inflammatories, non-steroidal anti-inflammatories, anti-neoplastics, anti-spasmodics, modulators of cell-extracellular matrix interactions, enzymes and enzyme inhibitors, anticoagulants and/or antithrombotic agents, DNA, RNA, inhibitors of DNA, RNA or protein synthesis, polypeptides, compounds modulating cell migration, compounds modulating proliferation and growth, and vasodilating agents.

8. The graft material of claim 1, wherein at least two therapeutic agents are present.

9. The graft material of claim 8, wherein the therapeutic agents are antibiotics, antivirals, and antifungals.

10. The graft material of claim 9, wherein the therapeutic agents are selected from the group consisting of penicillins, cephalosporins, cycloserine, vancomycin, imidazole antifungal agents, polymyxin, amphotericin B, chloramphenicol, tetracyclines, rifampin, erythromycin, clindamycin, rifamycins, quinolones, sulfonamides, zidovudine, acyclovir, and minocycline.

11. The graft material of claim 1, wherein the at least one therapeutic agents is released into a tissue in need thereof over time.

12. The graft material of claim 1, further comprising an adjuvant.

13. The graft material of claim 1, further comprising an additive.

14. The graft material of claim 13, wherein the additive is selected from the group consisting of stabilizers, fillers, antioxidants, catalysts, plasticizers, pigments, and lubricants.

15. A method for promoting healing of tissues, comprising:
contacting a tissue in need thereof with a graft material comprising an extracellular matrix (ECM) and at least one therapeutic agent.

16. The method of claim 15, wherein the tissue is skin.

17. The method of claim 15, wherein the ECM is selected from the group consisting of small intestine submucosa, acellular dermis, cadaveric fascia, the bladder acellular matrix, and amniotic membrane.

18. The method of claim 17, wherein the ECM is small intestine submucosa.

19. The method of claim 18, wherein the small intestine submucosa is fluidized.

20. The method of claim 15, wherein the therapeutic agent is selected from the group consisting of growth factors, antibiotics, antivirals, steroidal anti-inflammatories, non-steroidal anti-inflammatories, anti-neoplastics, anti-spasmodics, modulators of cell-extracellular matrix interactions, enzymes and enzyme inhibitors, anticoagulants and/or antithrombotic agents, DNA, RNA, inhibitors of DNA, RNA or protein synthesis, polypeptides, compounds modulating cell migration, compounds modulating proliferation and growth, and vasodilating agents.
